(19) United States
(12) Patent Application Publication
(10) Pub. No.: US 2017/0105916 A1
(43) Pub. Date: Apr. 20, 2017

(54) SUNLESS TANNING COMPOSITIONS COMPRISING CARAMEL

(71) Applicant: Australian Gold, LLC, Indianapolis, IN (US)

(72) Inventors: MAXINE TRUAX JOHNSON, Arizona City, AZ (US); Stacey Dahmen, Indianapolis, IN (US); Ashley M. Wottring, Indianapolis, IN (US); Lena J. Lindemann, Carmel, IN (US); Angie H. Provo, Carmel, IN (US); George F. Feldman, III, Indianapolis, IN (US)

(21) Appl. No.: 15/298,791

(22) Filed: Oct. 20, 2016

Related U.S. Application Data
(60) Provisional application No. 62/243,716, filed on Oct. 20, 2015.

(51) Int. Cl. A61K 8/35; A61Q 17/04; A61K 8/97

(52) U.S. Cl. CPC A61K 8/35; A61K 8/97; A61Q 17/04; A61K 2800/592

(57) ABSTRACT

Sunless tanning compositions and methods for using the same are described herein. Such compositions may include sunless tanning ingredients such as dihydroxyacetone. Additional colorants may also be included. These additional colorants are preferably caramel colorants, particularly those in Class IV such as DSL4. It was found that the additional coloring enhancers of the present disclosure unexpectedly contributed to the immediate and long-term color development of a suntanned appearance on skin even after these water-soluble colorants were washed off of skin.

### CROSS REFERENCE TO RELATED APPLICATIONS

The present application claims the right of priority to U.S. Provisional Patent Application No. 62/243,716, filed on Oct. 20, 2015, the entirety of which is incorporated herein by reference.

### FIELD OF THE DISCLOSURE

The present disclosure is in the field of sunless tanning compositions.

### BACKGROUND

Sunless tanning compositions are a popular cosmetic product. For example, sunless tanning compositions can impart color to the skin of a human without or with reduced exposure to sun or artificial UV light. Many people use sunless tanning compositions to feel good and to project the image of a healthy or active lifestyle. The resulting skin color may help them look and feel healthier and thinner.

Often, sunless tanning compositions are formulated from a variety of ingredients including those called “bronzers.” Some sunless tanning compositions may suffer from uneven color distribution on skin as well as coloring that may not aesthetically enhance the best preferred color of naturally tanned skin for a variety of skin types and/or skin tones.

Sunless tanning compositions typically last for only a limited period of time, for example, a few days. The time the color lasts on the skin may be reduced by many factors including, but not limited to, rubbing of the skin, washing of the skin, and/or unfavorable conditions of the skin such as dry skin.

Sunless tanning compositions may be applied by a variety of methods. Application methods include spraying a sunless tanning composition on the skin of a human by automated machine or human operated machine (e.g., air brush sprayer or pump sprayer), or by manually spreading creams or lotions over the skin.

In light of the background above, there remains a need for commercial sunless tanning compositions with improved color and longevity performance.

### SUMMARY

In one aspect, the present disclosure relates to improved compositions and methods for the sunless tanning of human skin.

In certain aspects, the present disclosure relates to the use of specific caramel colorants to enhance the color imparting characteristics of sunless tanning compositions. In certain embodiments, unexpected benefits have been found by selecting ingredients in useful proportions from the specific subclass of caramels classified as Class IV caramels. In particular, it has been discovered that the incorporation of the Class IV caramel known as DSL4 provides superior performance in imparting a desired skin color from sunless tanning compositions in both immediate and long term results. In certain embodiments, the immediate or long term effects of sunless tanning compositions containing DSL4 can be selectively enhanced by incorporating additional agents such as the caramels SC105 and P600.

In further embodiments, it has been found that the sunless formulations can be combined in proportions with mixtures of extracts made from beet, rosemary, turmeric, annatto, saffron, purple sweet potato, and/or carrot to affect the immediate and long term hue and/or tonality on the skin of the resulting sunless solution skin coloring. Other examples of extracts include cochineal, wheat, corn, pepper, spirulina, chlorophyll, red cabbage, grape skin. Extracts are water soluble and are normally expected to wash-off or rinse off the skin rather than having a long term effect.

In still another aspect, the present disclosure relates to the use of caramel colorants that comprise a low level of 4-methylimidizole (4-MEI), which may be a byproduct of caramel production.

In yet another aspect, embodiments of the present disclosure employ the use of one or more sunless tanning ingredients sometimes referred to as delayed tanning agents including, but not limited to, dihydroxyacetone, erythrulose, henna and black walnut.

In still other embodiments, dyes may be used as additional colorants in compositions of the present disclosure. For example, but not limited to, Red 4, Red 33, Red 40, Carmine, Blue 1, Yellow 5, and/or carbon black may be used in embodiments of the present disclosure.

In a further embodiment, one or more additional caramels may be present in formulations in addition to the one or more class IV caramels mentioned above. For example, it has been found that using a class IV caramel in combination with a class I caramel may improve the immediate and/or long term color of the sunless tanning composition on skin.

Additional embodiments of the disclosure, as well as features and advantages thereof, will be apparent from the descriptions herein.

### BRIEF DESCRIPTION OF THE DRAWINGS

FIG. 1 shows color grading using a forced ranking of various formulations in example 1 of sunless tanning compositions comprising caramel on skin immediately after application to skin.

FIG. 2 shows color grading using a forced ranking of various formulations in example 1 of sunless tanning compositions comprising caramel on skin after 24 hours.

FIG. 3 shows a bar graph of results of grading color immediately after application in example 2.

FIG. 4 shows a bar graph of results of grading color 24 hours after application in example 2.

FIG. 5 shows a bar graph of results grading solutions in example 3 with a caramel added to a sunless solution comprising DSL4 immediately after application.

FIG. 6 shows a bar graph of results grading solutions in example 3 with a caramel added to a sunless solution comprising DSL4 24 hours after application.

FIG. 7 shows a digital color image of skin color immediately after the application comparing the tint and hue of solutions comprising extracts to control solutions in example 4.

FIG. 8 shows a digital color image of skin color 24 hours after application comparing the tint and hue of solutions comprising extracts to control solutions in example 4.

### DETAILED DESCRIPTION

For the purposes of promoting an understanding of the principles of the disclosure, reference will now be made to certain embodiments and specific language will be used to describe the same. It will nevertheless be understood that no limitation of the scope of the disclosure is thereby intended, such alterations and further modifications, and such further applications of the principles of the disclosure as described herein being contemplated as would normally occur to one skilled in the art to which the disclosure relates.

As used herein, the term “immediately upon application” or “immediate” generally refers to a time period significantly less than 24 hours, including, for example 15-30 minutes after application. As used herein, the term “long term” and/or “over time” generally refers to a time period greater than about 24 hours, preferably at least about three to about five days, and more preferably at least about five days to about seven days.

While certain ingredients produced from carbohydrates are broadly classified as “caramels,” there are various types of caramels with substantial variations among and between the various types of caramels. The characteristics of the caramels may vary based on the particular class of the caramel and even among caramels within the same class. For example the caramels may vary in charge and/or pH in solution. When combined with other ingredients the caramels may have different properties. When incorporated with cosmetics applied to human skin, the different effects are further pronounced due to the different manners in which the different caramels interact with different skin types. It is also desirable to have caramels which are low in 4-Methylimidazole.

In certain embodiments, the present disclosure relates to improved compositions and methods for the sunless tanning of human skin. It has been found, unexpectedly, that certain caramel colorants perform better in improving both immediate and long term color provided by sunless tanning compositions with delayed tanning agents than others in different classes of caramels, and even within the same class of caramel colorants. In selected embodiments, it has been found that in useful proportions a low 4-MEI caramel colorant classified in Class IV, namely caramel DSL4 (Sethness Products Company, Skokie, Ill.), outperforms other caramel colorants in imparting color to skin both immediately and long term. Other Class IV caramels sold by Sethness Products Company that may be used in certain embodiments include RT120, SB115, AP150, AP100, TR240, BC 145, HPH 400, STDXX, SBDS, DS400, specifically including S190, RTL4 and LF363 as low 4-MEI caramels. Other Class IV caramels that may be used in embodiments of the present disclosure include those sold by DD Williams (Louisville, Ky.) such as product numbers 050, 055, 065, 105, 108, 111, 112, 135, 170, 173, 600, 602, 603, 605, 607, 608, 610, 663. In certain embodiments, Class I and/or Class III caramels may be used to enhance the results of the Class IV caramels. Class I and class III caramels that may be used include, for example, but are not limited to P600 and/or SC105 from Sethness.

Embodiments of the present disclosure may contain one or more delayed tanning agents. Delayed tanning agents typically develop a skin color over time after application rather than imparting an immediate color. Examples of suitable delayed tanning agents include, but are not limited to, dihydroxyacetone (“DHA”), erythrulose, and/or melanin. Delayed tanning agents may be used in embodiments of the present disclosure in combination with other tanning agents or ingredients.

One or more delayed tanning agents may be present in compositions of the present disclosure. For example, a combination of dihydroxyacetone and erythrulose may be used. In certain embodiments, two, three, four, five, six, or more delayed tanning agents may be used in combination with each other. The ratio of one delayed tanning agents to the other or others may be any suitable ratio. In one embodiment of the present disclosure, dihydroxyacetone is present in the ratio of about 150 to about 1 by weight of erythrulose. In another embodiment dihydroxyacetone is present in the ratio of about 130 to about 1 by weight of erythrulose. In still another embodiment, dihydroxyacetone is present in the ratio of about 75 to about 1 by weight of erythrulose. In yet another example, dihydroxyacetone is present in a ratio of about 1 to about 1 by weight of erythrulose.

In certain embodiments, dihydroxyacetone (DHA) may be present between about 0.1% by weight and about 20% by weight of the composition, between about 5% and 20% by weight, between about 13-19% by weight of the composition, and between about 13-15% by weight of the composition.

The color as well as other traits of human skin can vary widely. For example, some individuals have a light skin tone while others have a darker skin tone. The skin of some individuals may have more pink, yellow, and/or green undertones present while others have less. Correspondingly, sunless solutions react differently with different skin types. Therefore, it may be desirable to have sunless tanning compositions with different shades of color and/or color intensity. Formulations may be color-balanced with ingredients that contribute differently to the color spectrum and formulations may be designed to match the natural tint of suntanned skinned by altering the ratios of ingredients or colorants. For example, formulations may be designed to produce a light, medium or dark suntanned appearance. The color intensity and/or color spectrum imparted to a particular user can be controlled by selecting certain colorants or color enhancers, in combinations among themselves and/or with other ingredients, to produce a desired net color value or color intensity such as close to that of naturally suntanned skin.

In certain embodiments, the present disclosure relates to the use of sunless tanning formulations incorporating caramel colorants. In other preferred aspects, the present disclosure especially relates to the use of caramels classified as Class IV caramels, and specifically the Class IV caramel DSL4. In other aspects, class IV caramels may be combined with class I caramels.

In still other embodiments, the present disclosure relates to the use of caramel colorants that comprise a low level of 4-methylimidizole (4-MEI), which may be a byproduct of caramel production. 4-MEI has been implicated by some as a possible carcinogen. The state of California has sought to reduce the use of ingredients that comprise 4-MEI in consumer products, including in carbonated beverages.

Caramels are often prepared by heating carbohydrates, in the presence of acids, alkalis, or salts. The chemical products of caramel production are varied and can change based upon the processes used to produce the caramel. Caramel colorants are commonly classified in the industry based upon the processes used to produce the caramel. These classes are summarized below in Table 1.

TABLE 1
Classes of caramel colorants.

| | INS | E | | Restrictions On | |
|---|---|---|---|---|---|
| Class | No. | Number | Description | Preparation | Common uses |
| I | 150a | E150a | Plain caramel, | No ammonium | Often used in |
| | | | caustic caramel, | or sulfite | whisky or |
| | | | spirit caramel; | compounds can | other high |
| | | | | be used during | proof |
| | | | | the preparation. | alcohols. |
| II | 150b | E150b | Caustic sulfite | Sulfite | Often used in |
| | | | caramel; | compounds can | cognac, |
| | | | | be used but no | sherry, or |
| | | | | ammonium | vinegars. |
| | | | | compounds can | |
| | | | | be present | |
| III | 150c | E150c | Ammonia | ammonium | Often used in |
| | | | caramel, baker's | compounds may | beer, sauces, |
| | | | caramel, | be used, but no | or |
| | | | confectioner's | sulfite | confections. |
| | | | caramel, beer | compounds may | |
| | | | caramel; | be present. | |
| IV | 150d | E150d | Sulfite ammonia | Both ammonium | Often used in |
| | | | caramel, acid- | compounds and | acidic |
| | | | proof caramel, | sulfite | environments |
| | | | soft-drink | compounds are | such as soft |
| | | | caramel; | present. | drinks. |

Class IV caramel colorants are produced from carbohydrates that are heated in the presence of both sulfite and ammonium compounds. In certain embodiments, the Class IV caramel colorants may have an isoelectric point between pH 0.5 and 2, and may carry a negative ionic charge above pH 2.

Table 2 summarizes the physical properties of DSL4 and its corresponding physical properties that may be used in various embodiments of the present disclosure. These physical properties include the classification of the caramel, the tinctorial power, the baume of the caramel, the specific gravity of the caramel, the density of the caramel, the pH of the caramel, the typical color intensity of the caramel, and/or the quantity of 4-MEI present in the caramel. Other class IV caramel colorants may be used in certain embodiments based on having one or more similar physical properties, such as a low 4-MEI concentration.

TABLE 2
Summary of physical properties of Class IV caramel colorants
used in certain embodiments of the present disclosure.

| Caramel Colorant | Physical Properties | |
|---|---|---|
| DSL4 (Sethness Products | Class | IV |
| Company of Skokie, IL) | Tinctorial Power, K_{0.56} | 0.370-0.410 |
| | Baume @ 60° F. | 30.7-31.7 |
| | Specific Gravity @ 60° F. | 1.268-1.280 |
| | Pounds per gallon @ 60° F. | 10.56-10.66 |
| | pH | 2.5-3.0 |
| | Typical Color Intensity | 0.230-0.258 |
| | 4-MEI | <30 ppm |

Other colorants may be used in embodiments of the present disclosure. Certain colorants are extracts such as from beet, rosemary, annatto, saffron, turmeric, turmeric root, purple sweet potato, cochineal, carrots, wheat, corn, pepper, spirulina, chlorophyll, red cabbage, and grape skin. Other colorants are dyes such as, but not limited to, Red 4, Red 33, Red 40, Carmine, Blue 1, Yellow 5, and/or carbon black.

In order to promote a further understanding of the present disclosure and its various embodiments, the following specific examples are provided. It will be understood that these examples are illustrative and not limiting.

### Example 1

### Panel Study—Forced Ranking

### Materials and Methods:

A panel study was conducted to test the immediate and 24-hour color of various sunless tanning compositions comprising caramels and/or other colorants. In this study, a forced ranking system was used to rank the color on skin of various formulations (1 being the lightest).

Each formulation was prepared according to procedures used by those of ordinary skill in the art with 13.5% dihydroxyacetone and 0.1% preservative combined with 4% of the caramel or mixture of caramels being tested. The control sample included no further colorants.

### Results:

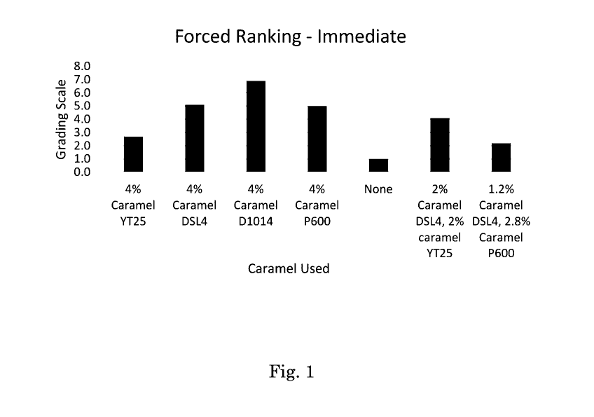

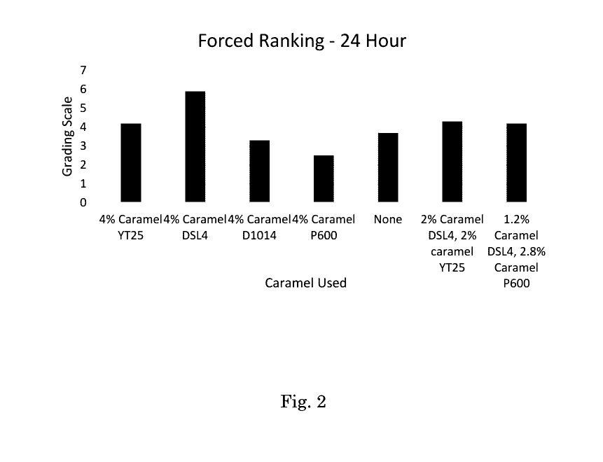

FIG. 1 shows the forced ranking of each formulation with regards to the color immediately upon application. FIG. 2 shows the forced ranking of each formulation with regards to the color after 24 hours.

In this study, the formulation comprising 4% of the caramel DSL4 showed the best balance of performance in both immediate color and 24 hour color. Caramels produced by various suppliers may have certain desirable attributes which may be attributed to the particular processes used to produce them. For example, different caramels may have varying amounts of electrolyte contributions, which may be obtained by different drying methods, such as where more or less ash or other drying agents can be left behind in the final caramel product.

### Example 2

### Panel Study

### Materials and Methods:

A panel study was conducted to evaluate various experimental sunless tanning solutions comprising caramel. Five solutions each comprising 4% by weight caramel and 13.5% by weight dihydroxyacetone, deionized water, and preservative were prepared. Caramels YT25 (a Class I caramel), DSL4 (a Class IV caramel), D1014 (a Class IV E150d caramel), P600 (a Class III caramel) and SC105 (a Class I caramel) were used in preparing these compositions. A control solution was also prepared comprising 13.5% dihydroxyacetone, deionized water, and preservative.

### Results:

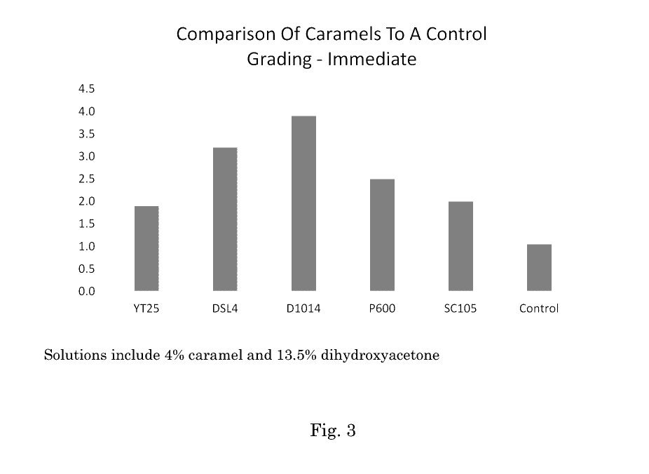

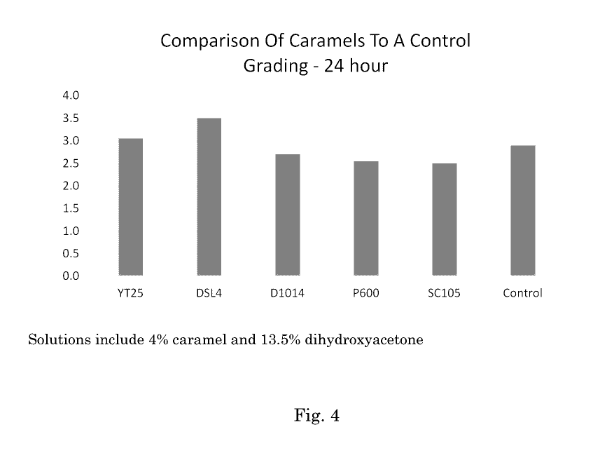

The caramel solutions generally speaking, as a whole, imparted some immediate color after application to skin. However, after 24 hours, DSL4, a Class IV caramel, scored higher than the control after 24 hours. FIG. 3 and FIG. 4 show a graphical representation of these data. From these data, it appears that although all caramels tested made an immediate impact on color development. D1014 and DSL4 scored the highest for immediate colors. Over time, the color imparted appeared to decline, however DSL4 maintained a score noticeably higher than the control after 24 hours. DSL4 contributed most consistently to both immediate and long term color. DSL4 was therefore chosen for use and further study.

### Example 3

### Panel Study

### Materials and Methods:

A panel study was conducted to evaluate a base sunless tanning solution comprising DSL4 complemented with a second caramel and/or other colorants. Five solutions were made each comprising 1.24% (w/w) DSL4 caramel, 13.5% (w/w) dihydroxyacetone, deionized water, preservative, and a second colorant. The control sample included no further colorants. The second colorants used, respectively, included: 0.23% P600 (w/w), 3.0% YT25 (w/w), and 5.43% SC105 (w/w). The percentages are different because the second colorants are supplied in different concentrations and/or there may be regulatory limits, such as MEI limits) on how much of a particular colorant can be used in a cosmetic product. The percentages of the second colorants were selected so that the solutions had a comparable/normalized color level prior to application to a person's skin.

Each solution was applied to the arm of each participant with 0.25 mL of solution in a circular manner. Immediate color was graded within 30 minutes of application. Color was also graded after 24 hours once participants had showered without exfoliation. Color was graded on a scale of 1 (light brown) to 5 (dark brown).

### Results:

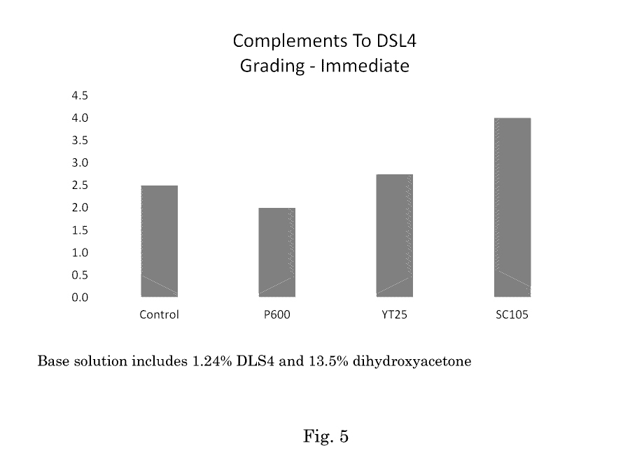

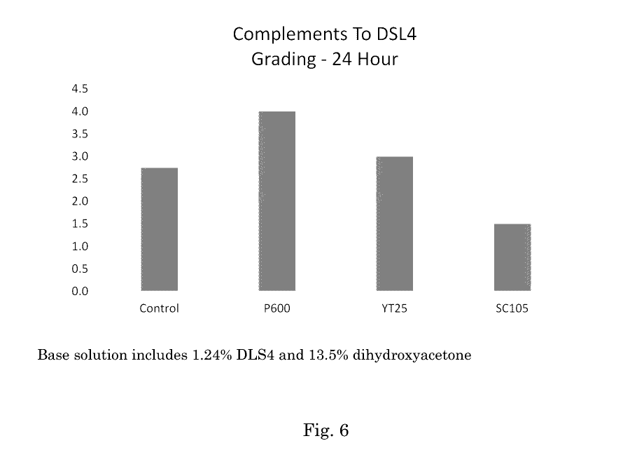

FIG. 5 and FIG. 6 show a graphical representation of the test data. Scores of 4 or 5 were considered to provide good color. Unexpectedly, the solution containing SC105 provided the best immediate color complement to the DSL4 sunless solution. Differently, the solution containing P600 provided the best long term color complement to the DSL4 sunless solution. Many users find immediate color to be important. From this data, the combination of DSL4 and SC105 provides superior immediate color. Alternately, some users of sunless tanning products find long term color more important. For longer term color, the combination of DSL4 and P600 provides superior results.

The weight percentages of the second colorants can be varied to adjust the immediate and long term resulting color of the solution before and after application to skin. For example, in certain embodiments P600 may be present between about 0.1% to 1.0 by weight of the composition, or between about 0.2% to 0.4% by weight. YT25 may be present between about 1.0% to 5.0% by weight of the composition, or between about 2.0% to 4.0%. SC105 may be present between about 1.0% to 10% by weight of the composition, optionally between about 3.0% to 6.0% by weight, and with specific examples between about 5.0% to 6.0% by weight.

### Example 4

### Panel Study

### Materials and Methods:

A panel study was conducted to evaluate the addition of extracts to various experimental sunless tanning solutions with and without DSL4 and with and without other dye colorants. “Extracts” includes a mixture of beet, rosemary, tumeric, annatto, saffron, purple sweet potato, and carrot extract, each of which was provided by Carrubba. The extracts are typically water soluble. Six compositions were prepared each using a base solution comprising 13.5% dihydroxyacetone, deionized water, and preservative. The compositions were compared in pairs.

Composition 1 comprised the base solution with dyes, namely 0.0145% FD&C Blue 1, 0.05014% FD&C Red 40, and 0.0121% FD&C Yellow 6, plus with 0.20% by weight of extracts added. Composition 2 comprised the base solution with the dyes of Composition 1 without the extracts.

Composition 3 comprised the base solution with the dyes of Composition 1 with 1.24% DSL4 caramel plus with 0.20% extracts added. Composition 4 comprised the base solution with the dyes of Composition 1 with 1.24% DSL4 caramel with no extracts added.

Composition 5 comprised the base solution with 1.24% DSL4 caramel plus 0.20% extracts added. Composition 6 comprised the base solution with 1.24% DSL4.

Each solution was applied to the arm of each participant with 0.25 mL of solution in a circular manner. Immediate color was photographed with digital images within 30 minutes of application. Color was also photographed with digital images after 24 hours after participants had showered without exfoliation.

### Results:

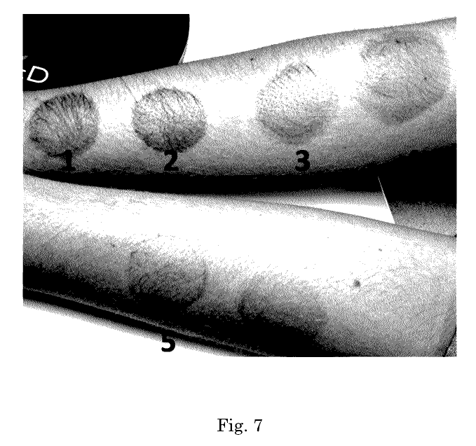

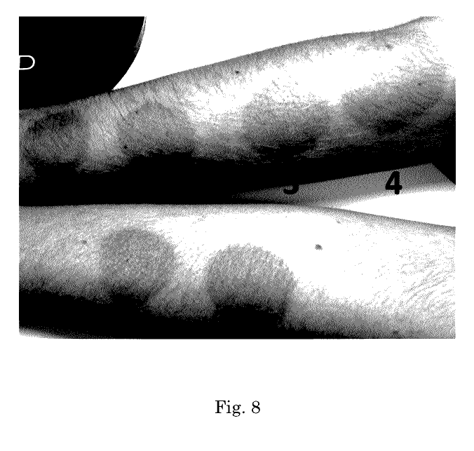

The resulting data, including digital images, are shown in FIG. 7 and FIG. 8. As can be seen from these images, sample 1 generally appears darker compared to sample 2 when observed immediately as well as when observed after 24 hours. Similarly, samples 3 and 5 generally appear darker compared respectively to samples 4 and 6 when observed immediately as well as when observed after 24 hours. Additionally, it was observed that the extracts contributed to differences in color tone and hue. The results illustrate that extracts can be useful to enhance and control the hue and/or tonality of the resulting sunless solution skin coloring both immediately and long term. These results, especially the long term results, are surprising since the extracts are water soluble and normally are expected to wash off in water. The combination of the extracts colorants with the sunless solution containing DSL4 expectantly appears to enhance the ability of the extracts to impart a long term color while also being used to control skin tone and hue.

The weight percentages and mixture of the extracts can be varied to adjust the immediate and long term resulting color of the solution before and after application to skin. For example, in certain embodiments the extracts may be present between up to about 2% by weight, or within a range of about 0.1% to 0.8% by weight of the composition. A specific example is 0.2% by weight.

In further embodiments, various mixtures combining the extracts can be used to provide a particular hue and/or tonality to the skin when applied to skin immediately as well as after 24 hours. The particular mixture may be selected to deliver a desired result, for example by matching a particular hue or tone to a particular skin type. The specific mixture of extracts may be chosen based upon the desired effect on the skin.

The results of these examples show that one of ordinary skill in the art may specifically use class IV caramels and particularly DSL4 when formulating sunless tanning solutions which fall within the scope of the claims appended hereto. The solution with DSL4 may be complimented with other caramels or extracts to selectively vary the resulting skin color both immediately and long term. Such caramels, whether alone, or in combination with other caramels or coloring agents may allow one of ordinary skill in the art to achieve the desired darkness, hue, and/or tonality of skin after application of a sunless tanning formulation.

Sunless tanning compositions according to any of the embodiments may comprise preservatives, one or more antioxidants, one or more pH modifiers, one or more pH buffer systems, one or more fragrances, one or more thickening agents, one or more emulsifying agents, one or more antifungal agents, one or more antimicrobial agents, one or more humectants, one or more emollients, one or more surfactants, one or more sunscreens, and/or one or more solvents. The total quantity of any one or more additives may be any suitable quantity as would be employed by those of ordinary skill in the art.

Various moisturizing agents or humectants that may be included in embodiments of sunless tanning compositions include, but are not limited to, amino acids, chondroitin sulfate, diglycerin, erythritol, fructose, glucose, glycerin, glycerol polymers, glycol, dimethyl isosorbide, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol, hydrogenated castor oil, PEG-40 hydrogenated castor oil, PEG-20 methyl glucose sesquistearate, PEG-40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

Embodiments of the disclosed sunless tanning compositions may be applied to the skin of a human in any suitable manner. Application may be performed by automated spray machine, human operated spray machine (e.g., air brush or pump spray), or by manually spreading creams or lotions over the skin.

Compositions of the present disclosure may be produced in any suitable formulation, including but not limited to, oil-in-water emulsions, lotions, liquid solutions, gels, creams, milks, foams, or sprays. Any suitable vehicle may be used in embodiments of the present disclosure, such suitable vehicles including cosmetically acceptable vehicles. Cosmetically acceptable vehicles may comprise, for example, but not limited to, water.

The stability of cosmetic formulations may be important for commercially significant reasons. For example, consumers may prefer a homogenous formulation that does not separate over time. Homogeneity may be measured by storing a formulation in a fluid-tight container for an amount of time and observing if phase separation occurs. For example a formulation preferably may be stored in a stationary state in a fluid-tight container for greater than one month, for greater than three months, for greater than six months, and/or for greater than 12 months.

The uses of the terms “a” and “an” and “the” and similar references in the context of describing the disclosure (especially in the context of the following claims) are to be construed to cover both the singular and the plural unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

While the disclosure has been illustrated and described in detail in the drawings and the foregoing description, the same is to be considered as illustrative and not restrictive in character, it being understood that only the preferred embodiment has been shown and described and that all changes and modifications that come within the spirit of the disclosure are desired to be protected. In addition, all references cited herein are indicative of the level of skill in the art and are hereby incorporated by reference in their entirety.

What is claimed is:

1. A sunless tanning composition comprising:
dihydroxyacetone between about 0.1 percent by weight and about 20 percent by weight; and
caramel between about 0.1 percent by weight and about 10 percent by weight; and
wherein said caramel is selected from class IV caramel colorants.

2. The sunless tanning composition of claim 1, comprising dihydroxyacetone between about 10.0 percent by weight and about 20.0 percent by weight.

3. The sunless tanning composition of claim 2, wherein said class IV caramel colorant is sold under the trade name DSL4 by Sethness Products Company.

4. The sunless tanning composition of claim 3, further comprising the caramel colorant sold under the trade name SC105 by Sethness Products Company.

5. The sunless tanning composition of claim 4, wherein the class I caramel colorant SC105 is present between about 3.0 percent and about 6.0 percent by weight of the composition.

6. The sunless tanning composition of claim 3, further comprising the class III caramel colorant sold under the trade name P600 by Sethness Products Company.

7. The sunless tanning composition of claim 6, wherein the caramel colorant P600 is present at about 0.1 percent to about 0.8 percent by weight of the composition.

8. The sunless tanning composition of claim 3, further comprising a mixture of extracts selected from the group consisting of extracts of beet, rosemary, tumeric, annatto, saffron, purple sweet potato, and carrot.

9. The sunless tanning composition of claim 8, wherein the mixture of extracts is between about 0.2 percent to about 2.0 percent by weight of the composition.

10. The sunless tanning composition of claim 2, wherein said class IV caramel colorant is present in between about 1.0 percent by weight and about 5.0 percent by weight of the composition.

11. The sunless tanning composition of claim 1, wherein said class IV caramel colorant has a 4-MEI of less than 30 ppm.

12. A sunless tanning composition comprising:
dihydroxyacetone between about 13 percent and about 15 percent by weight;
DSL4 class IV caramel colorant between about 0.1 percent and about 10 percent by weight; and,
SC105 caramel colorant between about 3.0 percent and about 6.0 percent by weight.

13. The sunless tanning composition of claim 12, wherein the DSL4 class IV caramel colorant is present in between about 0.1 percent by weight and about 5 percent by weight of the composition.

14. The sunless tanning composition of claim 12, wherein the DSL4 class IV caramel colorant is present at about 1.0 percent by weight.

15. The sunless tanning composition of claim 12, wherein the DSL4 class IV caramel colorant is present at about 4.0 percent by weight.

16. A sunless tanning composition comprising:
dihydroxyacetone between about 13 percent and about 15 percent by weight;
total caramels between about 0.1 percent by weight and about 10 percent by weight; and
wherein said at least one of said caramels is DSL4 class IV caramel colorants.

17. The sunless tanning composition of claim 16, wherein said class IV caramel colorant is present in between about 1.0 percent by weight and about 5.0 percent by weight of the composition.

18. The sunless tanning composition of claim 17, further comprising a class I or a Class III caramel.

19. The sunless tanning composition of claim 18, wherein said class I caramel comprises SC105.

20. The sunless tanning composition of claim 18, wherein said class III caramel comprises P600.
